(19) United States
(12) Patent Application Publication
(10) Pub. No.: US 2011/0195410 A1
(43) Pub. Date: Aug. 11, 2011

(54) Compositions and methods related to solid phase sequence detection and genotyping

(75) Inventor: Robert E. Wagner, JR., Nunn, CO (US)

(73) Assignee: GENE CHECK, INC., Greeley, CO (US)

(21) Appl. No.: 12/928,259

(22) Filed: Dec. 7, 2010

Related U.S. Application Data
(60) Provisional application No. 61/267,296, filed on Dec. 7, 2009.

(51) Int. Cl. C12Q 1/68

(52) U.S. Cl. 435/6.11

(57) ABSTRACT

The present invention provides improved compositions and methods of sequence detection and single nucleotide (“SNP”) genotyping. The methods of the present invention are related to combining sequence or allelic specific ligation on a solid phase platform with specific and efficient solid phase signal amplification. The methods for sequence detection include a first, second and third oligonucleotide. The methods of SNP genotyping include four oligonucleotides for SNPs associated with two alleles and additional oligonucleotides may be added for SNPs associated with more than two alleles. The usefulness of the present method is that it results in the determination of thousands of genotypes simply, rapidly and inexpensively on a solid support from a single genomic DNA sample.

### FIELD OF THE INVENTION

The present invention relates to the field of molecular biology and nucleic acid chemistry. More specifically, it relates to methods and reagents for identification of particular sequences, including Small Nucleotide Polymorphisms (“SNPs”), for a wide-variety of purposes including diagnostic, therapeutic, forensic and identity genotyping applications.

### BACKGROUND OF THE INVENTION

Recent and ongoing advances in sequencing technologies, particularly in the area of massively parallel sequencing, have facilitated the production of enormous amounts of sequence data from single genomic DNA samples. Currently available systems from Roche/454 (Genome Sequencer FLX—www.454.com), Applied Biosystems (SOLiD System—www3.appliedbiosystems.com) and Illumina (Genome Analyzer—www.illumina.com) allow as much as 60 GB of sequence to be obtained from a single sample. Perhaps the most powerful of these systems, the Genome Analyzer system of Illumina, allows output as high as 33 GB of sequence from a single sample and allows up to 96 fold multiplexing in a single run. This tremendous amount of sequence data output allows for greater than five fold coverage of the entire diploid human genome, and allows up to 96 fold multiplexing in a single run. So-called “third generation” sequencing, such as a system being developed by Pacific Biosciences, will allow single molecule, real time sequencing and may significantly reduce the actual time required for whole genome sequencing.

Massively parallel sequencing is of enormous value to current research applications, but will be of limited value for those applications requiring analysis of large numbers of samples, as is required for future diagnostic, therapeutic, forensic and identity genotyping applications. The sequencing protocols required for these applications are extremely complex and require expensive instrumentation and, unfortunately, the current massively parallel sequencing systems do not meet these criteria as they are relatively slow and not capable of running sophisticated protocols. For example, the Genome Analyzer system requires about 10 days to generate 33 GB of sequence, which equates to about 10 samples per day. This speed is not fast enough for most diagnostic, therapeutic, forensic or identity genotyping applications. Additionally, data analysis for such “re-sequencing” applications requires expensive software and considerable time for analysis. Given that most diagnostic, therapeutic, forensic and identity genotyping applications require examination of at most a few hundred or a few thousand specific nucleotides and that even whole genome mapping requires examination of only a few tens of thousands of nucleotide sequences, it appears for most clinical applications, massively parallel sequencing generates too much information too slowly to be a useful sequencing method.

A system that can quickly and inexpensively determine genotypes of up to several thousand single nucleotide polymorphisms will be helpful for purposes of current, as well as future, diagnostic, therapeutic, forensic and identity genotyping applications. All of these applications require a sequencing system that is capable of working directly with genomic DNA without the need for PCR or other amplification of individual sites of interest, high order multiplexing (>1,000 or >10,000 for mapping applications) and high throughput potential. They also require a simple and robust system to allow for clinical use, as well as have to be quick and low cost. The invention described herein fits these criteria.

### SUMMARY OF THE INVENTION

The present invention provides a method of specific sequence detection in a DNA sample that includes the following steps:
a. a first oligonucleotide, wherein at least a portion of the first oligonucleotide is complementary to a portion of a specific DNA sequence, wherein the first oligonucleotide is immobilized to a solid support such that the 3′ end of the first oligonucleotide is available to anneal with complementary DNA in the target DNA sample;
b. a second oligonucleotide, wherein at least of portion of the 5′ end is complementary to the specific DNA sequence immediately adjacent to the region to which the first oligonucleotide is complementary;
c. a third oligonucleotide immobilized to a solid support in the vicinity of the first oligonucleotide and at least a portion of the third oligonucleotide is complementary to a portion at or near the 3′ end of the second oligonucleotide;
d. annealing the second oligonucleotide to the third oligonucleotide before or after immobilization of the third oligonucleotide;
e. exposing the first and second oligonucleotides to the DNA sample under conditions that allow simultaneous annealing of the first and second oligonucleotides to a complementary strand of DNA in the DNA sample; and wherein ligation of said first and second oligonucleotides is diagnostic of the presence of the specific sequence in the DNA sample.

The ligation product may be amplified by the following procedure:
a. removing all oligonucleotides and DNA molecules from said solid support except those directly bound to said solid support;
b. allowing the 3′ end of said ligation products to anneal to said third oligonucleotide; and
c. extending said third oligonucleotide by DNA polymerase to copy said ligation product.

The ligation product may be further be subjected to:
a. denaturation of the double stranded product of said extension;
b. allowing the strands thus created to anneal to said first and third oligonucleotides; and
c. extending said first and third oligonucleotides by DNA polymerase to copy said strands.

The ligation product may be subjected to denaturation, annealing of the first and third oligonucleotides and extension of the first and third oligonucleotides multiple times.

The first and second oligonucleotides may be 50 to 100 nucleotides in length.

The third oligonucleotide may be 20 to 40 nucleotides in length.

The DNA sample may be selected from the group consisting of eukaryotic genomic DNA, prokaryotic genomic DNA, viral genomic DNA, plasmid DNA, restriction enzyme fragmented DNA, PCR amplicons and single stranded DNA.

The extension reaction may be accomplished with one or more labeled deoxynucleotide triphosphates. The label may be selected from the group consisting of radioactive, fluorescent, colorimetric, antigenic and enzymatic.

The present invention also provides a method of single nucleotide polymorphism (SNP) genotyping in a DNA sample that includes:
a. a first oligonucleotide complementary to a specific DNA sequence that includes the SNP immobilized to a solid support such that the 3′ end of the first oligonucleotide is available to anneal with complementary DNA at or one base adjacent to the SNP in a target DNA sample;
b. a second oligonucleotide, wherein at least of portion of the 5′ end is complementary to the specific DNA sequence that includes the SNP immediately adjacent to the region to which said first oligonucleotide is complementary;
c. a third oligonucleotide immobilized to a solid support in the vicinity of said first oligonucleotide, wherein at least a portion of said third oligonucleotide is complementary to a sequence at or near the 3′ end of said second oligonucleotide;
d. annealing said second oligonucleotide to said third oligonucleotide;
e. exposing the first and second oligonucleotides to the DNA sample under conditions that allow simultaneous annealing of the first and second oligonucleotides to a complementary strand of DNA in the DNA sample such that, when a specific allele containing the SNP is present in the DNA sample, the first and second oligonucleotides anneal to adjacent bases in the sample DNA, wherein ligation of the first and second oligonucleotides is diagnostic of the presence of said SNP in said DNA sample.

The ligation product may be amplified by the following procedure:
a. removing all oligonucleotides and DNA molecules from said solid support except those directly bound to said solid support;
b. allowing the 3′ end of said ligation products to anneal to said third oligonucleotide; and
c. extending said third oligonucleotide by DNA polymerase to copy said ligation product.

The ligation product may be further be subjected to:
a. denaturation of the double stranded product of said extension;
b. allowing the strands thus created to anneal to said first and third oligonucleotides; and
c. extending said first and third oligonucleotides by DNA polymerase to copy said strands.

The ligation product may be subjected to denaturation, annealing of the first and third oligonucleotides and extension of the first and third oligonucleotides multiple times.

The first and second oligonucleotides may be 50 to 100 nucleotides in length.

The third oligonucleotide may be 20 to 40 nucleotides in length.

The DNA sample may be selected from the group consisting of eukaryotic genomic DNA, prokaryotic genomic DNA, viral genomic DNA, plasmid DNA, restriction enzyme fragmented DNA, PCR amplicons and single stranded DNA.

The extension reaction may be accomplished with one or more labeled deoxynucleotide triphosphates. The label may be selected from the group consisting of radioactive, fluorescent, colorimetric, antigenic and enzymatic.

### DEFINITIONS

For the purpose of the present invention, the following terms shall have the following meanings:

“Allele” has the meaning which is commonly known in the art, that is, a genomic variant of a referent gene, including variants, which, when translated result in functional or dysfunctional (including non-existent) gene products. The first identified allelic form is arbitrarily designated as the reference form and other allelic forms are designated as alternative or variant alleles.

“For the purpose of determining genotype” means that one of the purposes is to determine genotype, not necessarily that the end goal or use of the information is to determine genotype. For instance, “for the purpose of determining genotype” includes the use of the information to determine genotype for the ultimate goal of determining a patient's probability of having a presusceptability to developing a particular disease.

“Gene” has the meaning that is commonly-known in the art, that is, a nucleic acid sequence that includes the translated sequences that code for a protein (“introns”) and the untranslated intervening sequences (“exons”), and any regulatory elements ordinarily necessary to translate the protein.

“Genotype” has the meaning that is commonly-known in the art, that is, a physical description of a nucleic acid sequence.

“Hybridization” has the meaning that is commonly-known in the art, that is, the formation of a duplex structure by two single-stranded nucleic acids due to complementary base pairing. Hybridization can occur between exactly complementary nucleic acid strands or between nucleic acid strands that contain some regions of mismatch.

“Nucleic acid” and “oligonucleotide” have the meaning that is commonly-known in the art, and include primers, probes, and oligomer fragments, and shall be generic to polydeoxyribonucleotides (containing 2-deoxy-D-ribose), to polyribonucleotides (containing D-ribose), and to any other type of polynucleotide which is an N glycoside of a purine or pyrimidine base, or modified purine or pyrimidine base. There is no intended distinction in length between the terms “nucleic acid” and “oligonucleotide”, and these terms will be used interchangeably. These terms refer only to the primary structure of the molecule. Thus, these terms include double- and single-stranded DNA, as well as double- and single-stranded RNA.

“Polymorphism” means one variant of a group of two or more nucleic acids.

“Sequence” means any series of nucleic acid bases or amino acid residues, and may or may not be a gene or encode a protein.

“Single nucleotide polymorphism” means a sequence variant involving only a single nucleotide.

Moreover, for the purpose of the present invention, the term “a” or “an” entity refers to one or more of that entity; for example, “a protein” or “a nucleic acid molecule” refers to one or more of those compounds or at least one compound. As such, the terms “a” (or “an”), “one or more” and “at least one” can be used interchangeably herein. It is also to be noted that the terms “comprising”, “including”, and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure, protein or nucleic acid molecule is a compound that has been removed from its natural millieu. As such, “isolated” and “biologically pure” do not necessarily reflect the extent to which the compound has been purified. An isolated compound of the present invention can be obtained from a natural source, can be produced using molecular biology techniques or can be produced by chemical synthesis.

### FIGURES

FIG. 1 illustrates sequence identification according to an embodiment of the present invention.

FIG. 2 illustrates the results of a genotyping experiment from QQ, QR and RR allele sheep according to an embodiment of the present invention.

### DETAILED DESCRIPTION OF THE INVENTION

The compositions and methods of the present invention are related to sequence detection and single nucleotide polymorphism (SNP) genotyping and combine sequence or allele specific ligation on a solid phase platform with specific and efficient solid phase signal amplification. Since there are no free oligonucleotides in the methods of the present invention and since each microarray spot functions completely independently of all other spots on the array, the present invention provides methods for virtually unlimited multiplexing with the only limit being the number of spots that can be applied to a solid support, such as a microarray slide. Thus, the methods of the present invention provide for the determination of thousands of genotypes simply, rapidly and inexpensively on a single slide from a single genomic DNA sample.

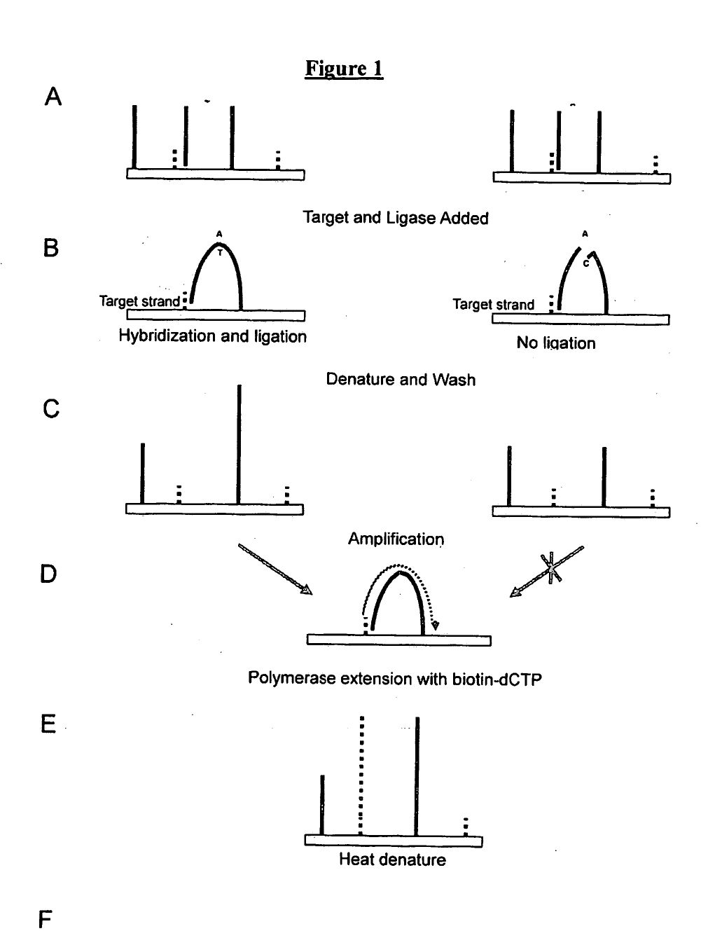

The methods of the present invention utilize three oligonucleotides for sequence identification. The methods of the present invention further utilize four short oligonucleotides for genotyping a two allele SNP (FIG. 1). Additional oligonucleotides can be utilized for genotyping SNPs associated with more than two alleles.

Any oligonucleotide may be utilized in the sequence identification or SNP genotyping methods of the present invention. One skilled in the art is familiar with creating and/or obtaining the oligonucleotides of the present invention.

A variety of the compositions and methods of the present invention relate to sequence identification. The sequence identification methods of the present invention utilize three oligonucleotides and any combination of any three oligonucleotides of any length and/or type may be utilized in the methods of the present invention.

In one embodiment, the three oligonucleotides utilized for sequence identification are short. For instance, the first and second oligonucleotides utilized for sequence identification are on the order of 50-90 bases long and contain homology to the target sequence being detected or genotyped.

The 5′ and/or the 3′ end of the first oligonucleotide utilized for sequence identification may be modified. In a particular embodiment, the 5′ end of the first oligonucleotide is modified, by adding an amine group to allow immobilization to a solid support, such as a microarray slide. In such a first oligonucleotide, it may also have 20-30 bases of sequence at its 3′ end that is not complementary to the target sequence. Any modification may be made to either one or both of the 5′ or 3′ end of the first oligonucleotide and one skilled in the art is familiar with choosing and then creating such modifications to this oligonucleotide.

A second oligonucleotide utilized for sequence identification may be complementary to the target sequence.

The 5′ and/or the 3′ end of the second oligonucleotide utilized for sequence identification may be modified. In a particular embodiment, the second oligonucleotide contains a 5′ phosphate group to allow ligation to the immobilized first oligonucleotide when both are correctly paired with the target sequence. In another particular embodiment, the terminal 20-30 nucleotides of the second oligonucleotide are designed to allow hybridization to an immobilized third oligonucleotide.

The 5′ and/or the 3′ end of the third oligonucleotide utilized for sequence identification may also modified. In a particular embodiment, the third oligonucleotide is modified at its 5′ end to allow immobilization. In another particular embodiment, the third oligonucleotide is modified at its 5′ end in such a way as to allow it to anneal to the second oligonucleotide so as to cause immobilization of the second oligonucleotide. Such annealing may occur as a result of modification and/or the sequence design of the terminal 20-30 nucleotides of the second oligonucleotide as to cause this annealing and subsequent immobilization to occur. One skilled in the art is familiar with designing oligonucleotides in such a way as to ensure both reactions occur.

Both the first and third oligonucleotides utilized for sequence identification may be immobilized. In a particular embodiment, both the first and third immobilized oligonucleotides will serve as primers for the amplification phase of the reaction. Any modification may be made to the 5′ or 3′ end of the first or third oligonucleotides to ensure such immobilization and one skilled in the art is familiar with choosing and then creating such modifications to one or both oligonucleotides.

Many of the compositions and methods of the present invention relate to SNP genotyping. The genotyping methods of the present invention utilize four oligonucleotides for a two allele SNP and additional oligonucleotides may be added for SNPs associated with more than two alleles. Any combination of four or more oligonucleotides may be utilized in the methods of the present invention and one skilled in the art is familiar with designing and producing such oligonucleotides.

In one embodiment, the first oligonucleotide utilized for SNP genotyping is modified at its 5′ or its 3′ end. In a particular embodiment, the modification is made at the 5′ end. In another particular embodiment, the modification is at the 5′ end for purposes of causing immobilization of the oligonucleotide. One skilled in the art is familiar with designing and producing an oligonucleotide capable of immobilization at its 5′ end. In another particular embodiment, the modification includes designing the terminal nucleotide of the first oligonucleotide in order for it to be allele specific. This may work to make the first oligonucleotide complementary to one allele of the target SNP.

In another embodiment, both the second and third oligonucleotides utilized for SNP genotyping are not allele specific. In other words, they are both “universal.”

In an additional embodiment, the second oligonucleotide utilized for SNP genotyping is designed to allow for immobilization by hybridization to a third, immobilized oligonucleotide. In a particular embodiment, the terminal 20-30 nucleotides of the second oligonucleotide are designed to immobilize the second oligonucleotide by causing it to anneal to the third oligonucleotide. In another particular embodiment, the third oligonucleotide is short and modified at its 5′ end to allow for covalent immobilization.

In another embodiment, both the first and third oligonucleotides will serve as primers for the amplification phase of the ILA reaction.

In an additional embodiment, the fourth oligonucleotide utilized for SNP genotyping is complementary to the target sequence.

In another embodiment, the fourth oligonucleotide utilized for SNP genotyping is modified at its 5′ end. In a particular embodiment, the modification includes a 5′ phosphate group. In another particular embodiment, the modification allows for ligation to the first oligonucleotide. In an additional particular embodiment, the fourth oligonucleotide anneals with the first oligonucleotide when both are correctly paired with the target sequence. In another particular embodiment, the first oligonucleotide is immobilized so such annealing causes both the first and fourth oligonucleotides to be immobilized when both are correctly paired with the target sequence.

In another embodiment, the fourth oligonucleotide utilized for SNP genotyping may include sequences that are not complementary to the target sequence. In a particular embodiment, the fourth oligonucleotide has 20-30 bases of sequence at its 3′ end that is not complementary to the target sequence.

In another embodiment, the fourth oligonucleotide utilized for SNP genotyping is identical to the first oligonucleotide, except for its terminal nucleotide. In a particular embodiment, the difference between the first and fourth oligonucleotide is that the terminal end of the fourth oligonucleotide is complementary to a different allele of the SNP than the terminal end of the first oligonucleotide.

In another embodiment, the first and third oligonucleotides utilized for SNP genotyping are immobilized. In a particular embodiment, such immobilization is covalent. In another particular embodiment, such immobilization occurs such that both the first and third oligonucleotides are in close proximity. In another particular embodiment, both the first and third oligonucleotides are located in a single microarray spot.

In an additional embodiment, the third and fourth oligonucleotides utilized for SNP genotyping are immobilized. In a particular embodiment, both the third and fourth oligonucleotides are immobilized. In another particular embodiment, they are immobilized in close proximity. In another particular embodiment, the third and fourth oligonucleotides are immobilized but not in close proximity to the first and third oligonucleotides.

In an embodiment where the SNP genotyping involves a SNP having more than two alleles, the additional oligonucleotides utilized will be almost identical to the first and fourth oligonucleotides except for the terminal nucleotides. One skilled in the art is familiar with designing oligonucleotides directed to different alleles of a particular SNP.

In another embodiment, both the first and third oligonucleotides utilized for SNP genotyping will serve as primers for the amplification phase of the ILA reaction. In a particular embodiment, both the first and third oligonucleotides are immobilized.

For purposes of utilizing the methods of the present invention for genotyping, target DNA, may be fragmented by any method known in the art. An illustrative method includes, but is not limited to, sonication. After fragmentation, the target DNA is denatured and allowed to hybridize to the immobilized oligonucleotides. In one embodiment, the immobilized oligonucleotides, which are targets for hybridization are the first, second and fourth oligonucleotides (FIG. 1B). In a particular embodiment, the first and fourth oligonucleotides are both allele-specific and are covalently immobilized. In another particular embodiment, the second oligonucleotide is also allele specific and immobilized by hybridization to the third oligonucleotide.

Hybridization is accomplished in the presence of a DNA ligase so that perfectly paired oligonucleotides hybridized to the same target strand will be ligated. Following hybridization and ligation, the solid support may be treated, by methods well known in the art, to denature and remove all DNA not covalently linked to the slide (FIG. 1C).

According to the methods of the present invention, if there is sequence present in the target DNA which is complementary to two of the immobilized oligonucleotides in the array spot, i.e., is the target sequence or, for SNP genotyping, is of the same allele as the array oligonucleotides, following exposure to ligase the array spot will contain at least a few molecules which are the product of ligation of oligonucleotides originally in the spot (FIG. 1C). If such a sequence or allele is not present in the target DNA, there will be no such ligated oligonucleotide products.

If ligation does occur, the methods of the present invention provide for amplification of the ligation products directly in the array spot. The 5′ end of the ligation product is complementary to the short immobilized oligonucleotide, which can serve as a primer to copy the entire ligation product (FIG. 1D). Multiple cycles of denaturation, annealing and extension can be performed using the immobilized oligonucleotides as primers (FIG. 1E-F). Labeled dNTPs can be incorporated during the amplification so that labeled spots will then indicate the presence of a specific allele in the target DNA sample.

Suitable solid supports for the methods of the present invention include, but are not limited to, microarray slides, flow cells, beads and microtiter plates. One skilled in the art is familiar with the identification and use of a variety of solid support systems.

### EXAMPLES

The following examples are included to demonstrate particular embodiments of the invention. It should be appreciated by those of skill in the art that the techniques disclosed in the examples which follow represent techniques discovered by the inventors to function well in the practice of the invention, and thus can be considered merely to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example One

In this example, genotyping was accomplished using the methods of the present invention directed to immobilized ligation and amplification. Target DNA was a 190 base PCR amplicon from the prion protein gene of sheep genomic DNA. The specific target SNP is located at the second position of codon 171.

Oligonucleotides applied to the microarray spot included the following:
1. A first allele specific oligonucleotide that was 85 bases long with an amine group at the 5′ end and a nucleotide at the 3′ end that was complementary to one allele of the target SNP. This allele specific oligonucleotide was immobilized to the microarray slide by means of the 5′ amine group.
2. A second “universal” 85 base oligonucleotide complementary to the target sequence where the 5′ nucleotide was phosphorylated to allow ligation to the first allele specific oligonucleotide when both oligonucleotides were paired with target DNA of the correct allele. This non-specific second “universal” oligonucleotide was immobilized to the slide, in the same spot as the allele specific oligonucleotide, by hybridizing to a third oligonucleotide complementary to 30 bases at the 3′ end of the second universal oligonucleotide sequence (FIG. 1) and immobilized by means of an amine group at its 5′ end.

A second allele-specific spot was prepared utilizing an allele-specific oligonucleotide (fourth oligonucleotide) specific for the second allele of the SNP and the same second and third oligonucleotides.

The PCR sample was denatured at 95° C. for 4 minutes before being moved to an ice bath. It was subsequently transferred to a slide subarray chamber that divided the slide into 10 subarrays. Each subarray contained two spots of each allele specific mix of the three oligonucleotides. Hybridization of the target DNA took place at 65° C. for 1 hour, followed by addition of T4 DNA ligase and further incubation at 37° C. for 30 minutes. The slides were then denatured and washed twice with sodium hydroxide to remove all target DNA and un-ligated universal oligonucleotides.

### Example Two

In this example, genotyping was accomplished using the methods of the present invention and limited manual thermal cycling was utilized to successfully demonstrate the potential of solid phase amplification for signal generation (FIG. 2).

A PCR amplicon (190 bp) of a portion of the Ovine prion protein gene was mixed with 1 ug human genomic DNA in 50 mM Tris, 1 mM MgCl2, 5 mM Spermidine, and 5 mM di-thiothreitol. 28 nmol ATP was added and the mixture was then denatured at 95° C. before being cooled rapidly on ice. The mixture was then added to an aldehyde slide divided into ten subarrays, each printed with two spots of the R probe and Q probe mixes.

The slides were then incubated for 1 hour at 65° C. before being cooled to 37° C. One unit of T4 DNA ligase was added and incubation continued for another 30 minutes. The slide was then washed in two 0.5M NaOH washes and rinsed in TNT. Subarray chambers were then reapplied to the slide, 75° C. water was added to each subarray and the slide chamber was then incubated on a 75° C. heating block for 10 minutes.

The extension mix of 1 u Tag polymerase and 0.75 nmol each of a dNTP mix was heated to 95° C. before being added to the subarrays. The slide chamber was then set at room temperature for 2 minutes prior to removal of the extension mixture and replacement by an extension mixture including 1 u Taq polymerase and 0.75 nmol each of dATP, dGTP and dTTP, and 0.375 nmol biotin-dCTP heated to 95° C. After 2 minutes the mix was removed and the extension mix, incubation and removal was repeated. The slides were then washed in NaOH and TNT as before, and developed for signal by the Perkin Elmer TSA-Cy3 kit. Slides were scanned at a gain of 100 on a Perkin Elmer ScanArray, and data displayed in FIG. 2 include the average of the median signal intensities minus background noise from the two spots per probe in each subarray.

Development of signal from these immobilized amplicons was with a Tyramide Cyanine-3 amplification kit (Perkin Elmer). The entire ILA reaction, from PCR amplicon to scanning for signal on a Perkin Elmer ScanArray took less than four hours.

As FIG. 2 indicates, successful genotyping of PCR product from QQ, QR and RR allele sheep was achieved from ligation through this simple method of three cycle amplification (FIG. 2). 1 ug of human genomic DNA was also present.

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of specific sequence detection in a DNA sample comprising:
a. a first oligonucleotide, wherein at least a portion of said first oligonucleotide is complementary to a portion of a specific DNA sequence, wherein said first oligonucleotide is immobilized to a solid support such that the 3′ end of said first oligonucleotide is available to anneal with complementary DNA in the target DNA sample;
b. a second oligonucleotide, wherein at least of portion of the 5′ end is complementary to said specific DNA sequence immediately adjacent to the region to which said first oligonucleotide is complementary;
c. a third oligonucleotide immobilized to a solid support in the vicinity of said first oligonucleotide and at least a portion of said third oligonucleotide is complementary to a portion at or near the 3′ end of said second oligonucleotide;
d. annealing said second oligonucleotide to said third oligonucleotide before or after immobilization of said third oligonucleotide;
e. exposing said first and second oligonucleotides to said DNA sample under conditions that allow simultaneous annealing of said first and second oligonucleotides to a complementary strand of DNA in said DNA sample; and wherein ligation of said first and second oligonucleotides is diagnostic of the presence of said specific sequence in said DNA sample.

2. The method of claim 1, wherein said ligation product is amplified by:
a. removing all oligonucleotides and DNA molecules from said solid support except those directly bound to said solid support;
b. allowing the 3′ end of said ligation products to anneal to said third oligonucleotide; and
c. extending said third oligonucleotide by DNA polymerase to copy said ligation product.

2. The method of claim 2, wherein said ligation product is further subjected to:
a. denaturation of the double stranded product of said extension;
b. allowing the strands thus created to anneal to said first and third oligonucleotides; and
c. extending said first and third oligonucleotides by DNA polymerase to copy said strands.

3. The method of claim 3, wherein said ligation product is further subjected to steps a, b and c multiple times.

4. The method of claim 1, wherein said first and second oligonucleotides are 50 to 100 nucleotides in length.

5. The method of claim 1, wherein said third oligonucleotide is 20 to 40 nucleotides in length

6. The method of claim 1, wherein said DNA sample is selected from the group consisting of eukaryotic genomic DNA, prokaryotic genomic DNA, viral genomic DNA, plasmid DNA, restriction enzyme fragmented DNA, PCR amplicons and single stranded DNA.

7. The method of claim 2, wherein said extension is accomplished with one or more labeled deoxynucleotide triphosphates.

8. The method of claim 8, wherein said label is selected from the group consisting of radioactive, fluorescent, colorimetric, antigenic and enzymatic.

9. A method of single nucleotide polymorphism (SNP) genotyping in a DNA sample comprising:
a. a first oligonucleotide complementary to a specific DNA sequence that includes said SNP immobilized to a solid support such that the 3′ end of said first oligonucleotide is available to anneal with complementary DNA at or one base adjacent to said SNP in a target DNA sample;
b. a second oligonucleotide, wherein at least of portion of the 5′ end is complementary to said specific DNA sequence that includes said SNP immediately adjacent to the region to which said first oligonucleotide is complementary;
c. a third oligonucleotide immobilized to a solid support in the vicinity of said first oligonucleotide, wherein at least a portion of said third oligonucleotide is complementary to a sequence at or near the 3′ end of said second oligonucleotide;
d. annealing said second oligonucleotide to said third oligonucleotide;
e. exposing said first and second oligonucleotides to said DNA sample under conditions that allow simultaneous annealing of said first and second oligonucleotides to a complementary strand of DNA in said DNA sample such that, when a specific allele containing said SNP is present in said DNA sample, said first and second oligonucleotides anneal to adjacent bases in said sample DNA, wherein ligation of said first and second oligonucleotides is diagnostic of the presence of said SNP in said DNA sample.

10. The method of claim 10, wherein said ligation product is amplified by:
a. removing all oligonucleotides and DNA molecules from said solid support except those directly bound to said solid support;
b. allowing the 3′ end of said ligation products to anneal to said third oligonucleotide; and
c. extending said third oligonucleotide by DNA polymerase to copy said ligation product.

11. The method of claim 11, wherein said ligation product is further subjected to:
a. denaturation of the double stranded product of said extension;
b. allowing the strands thus created to anneal to said first and third oligonucleotides; and
c. extending said first and third oligonucleotides by DNA polymerase to copy said strands.

12. The method of claim 12, wherein said ligation product is further subjected to steps a, b and c multiple times.

13. The method of claim 10, wherein said first and second oligonucleotides are 50 to 100 nucleotides in length.

14. The method of claim 10, wherein said third oligonucleotide is 20 to 40 nucleotides in length.

15. The method of claim 10, wherein said DNA sample is selected from the group consisting of eukaryotic genomic DNA, prokaryotic genomic DNA and viral genomic DNA, plasmid DNA, restriction enzyme fragmented DNA, PCR amplicons and single stranded DNA.

16. The method of claim 10, wherein said extension is accomplished with one or more labeled deoxynucleotide triphosphates.

17. The method of claim 15, wherein said label is selected from the group consisting of radioactive, fluorescent, colorimetric, antigenic and enzymatic.
